(19) United States
(12) Patent Application Publication
(10) Pub. No.: US 2009/0139433 A1
(43) Pub. Date: Jun. 4, 2009

(54) DENTAL MATERIALS BASED ON ALKYLENEDIAMINE-N,N,N',N'- TETRAACETIC ACID- (METH)ACRYLAMIDES

(75) Inventors: Norbert MOSZNER, Triesen (LI); Jorg ANGERMANN, Sargans (CH); Urs Karl FISCHER, Arbon (CH); Volker M. RHEINBERGER, Vaduz (LI)

Correspondence Address: NIXON PEABODY LLP - PATENT GROUP, 1100 CLINTON SQUARE, ROCHESTER, NY, 14604, US

(73) Assignee: Ivoclar Vivadent AG, Schaan (LI)

(21) Appl. No.: 12/207,622

(22) Filed: Sep. 10, 2008

(30) Foreign Application Priority Data
Nov. 30, 2007 (EP) 07023228.5

(51) Int. Cl. C08L 33/26; C07C 233/20; A61K 6/00; C07C 229/04; C08F 22/38

(52) U.S. Cl. 106/35; 562/565; 526/304; 522/175; 523/118; 523/116; 522/83; 560/169

(57) ABSTRACT

The present invention relates to an alkylenediamine-N,N,N′,N′-tetraacetic acid-(meth)acrylamide of formula (I)

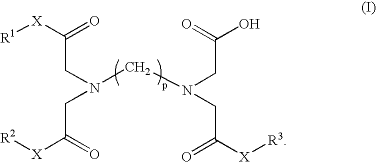

The invention also relates to a polymerizable composition which is characterized in that it comprises at least one alkylenediamine-N,N,N′,N′-tetraacetic acid-(meth)acrylamide of formula (I), and its use in particular as dental material.

The present invention relates to compositions based on alkylenediamine-N,N,N′,N′-tetraacetic acid-(meth)acrylamides which are characterized by a high resistance to hydrolysis and are particularly suitable as dental materials. The invention also relates to alkylenediamine-N,N,N′,N′-tetraacetic acid-(meth)acrylamides and the use thereof.

Monofunctional amides of acrylic or methacrylic acid are used in various fields of dentistry. Thus various acryl- or methacrylamides have been proposed as comonomers for the preparation of polymers for prostheses (GB 1 039 750). Polyfunctional (meth)acrylamides, e.g. bisacrylamides, are described as a suitable crosslinking component for dental adhesives or composites i.a. in US 2002/014138 A1 and WO 02/13768 A2. Furthermore suitably functionalized acryl- or methacrylamides are suitable as effective bonding components or comonomers in dental adhesives, primarily carboxyl-group-containing (methacrylamides, such as e.g. N-acryloylaspartic acid (Y. Totti et al., Dental Materials 19 (2003) 253-258) or N-methacryloyl-4-aminovaleric acid (N. Nishiyama et al., Biomaterials 25 (2004) 5441-5447), and also hydroxyl-group-containing (meth)acrylamides, such as e.g. N-(2-hydroxyethyl)-N-methylacrylamide, the use of which is described in EP 1 374 828 A1.

Chelate complex formation of ethylenediaminetetraacetic acid (EDTA) and the great affinity of the disodium salt of EDTA for calcium ions is known from complexometry (cf. RÖMPP Lexikon Chemie, 10^{th} ed., Georg Thieme Verlag, Stuttgart and New York, 1997, p. 2221). Adhesive compositions based on (meth)acrylic acid ester derivatives of EDTA are described in DE 10 2005 022 172 A1, but these adhesion-promoter components are characterized by a poor resistance to hydrolysis.

The object of the invention is to provide dental materials which are polymerizable, are characterized by a good solubility and display an improved storage stability in the presence of water. In addition, the materials are to display a good adhesion to dentin and tooth enamel.

The object is achieved according to the invention by a polymerizable composition comprising at least one alkylenediamine-N,N,N′,N′-tetraacetic acid-(meth)acrylamide of formula (I)

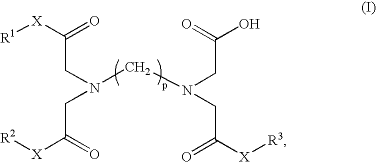

wherein
X represents in each case independently 0 or preferably NR⁶,
R¹, R² and R³ independently of one another represent hydrogen
   or

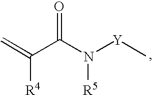

wherein R¹, R² and R³ are not simultaneously hydrogen,
Y represents in each case independently a linear or branched C₁ to C₁₅ alkylene radical which can be interrupted by one or more O or S,
R⁴ represents in each case independently hydrogen or a C₁ to C₁₀ alkyl radical,
R⁵ represents in each case independently hydrogen or a C₁ to C₈ alkyl radical,
R⁶ represents in each case independently hydrogen or a C₁ to C₆ alkyl radical and
p is 2 to 10.

The indication that a radical can be interrupted by a heteroatom such as O or S is to be understood such that the heteroatoms are inserted into the carbon chain of the radical, i.e. are bordered on both sides by carbon atoms. The number of heteroatoms is therefore at least 1 less than the number of carbon atoms, and the heteroatoms cannot be terminal.

A preferred embodiment of the polymerizable composition is characterized in that
Y represents in each case independently a linear or branched C₁ to C₁₀ alkylene radical which can be interrupted by one or more O or S,
R⁴ represents in each case independently hydrogen or a C₁ to C₈ alkyl radical,
R⁵ represents in each case independently hydrogen or a C₁ to C₆ alkyl radical,
R⁶ represents in each case independently hydrogen or a C₁ to C₄ alkyl radical and
p is 2 to 6.

Y particularly preferably represents a linear or branched C₁ to C₆ alkylene radical, in particular a linear or branched C₂ to C₅ alkylene radical, most preferably an ethylene radical.

R⁴ particularly preferably represents hydrogen or a C₁ to C₄ alkyl radical, in particular hydrogen or a methyl radical.

R⁵ particularly preferably represents hydrogen or a C₁ to C₄ alkyl radical, in particular hydrogen or a methyl radical.

R⁶ particularly preferably represents hydrogen or a C₁ to C₃ alkyl radical, in particular hydrogen or a methyl radical.

p is particularly preferably 2 to 4, most preferably 2.

The named alkyl and alkylene radicals are preferably linear groups.

It is particularly preferred that R¹ represents hydrogen and R² and R³ in each case represent

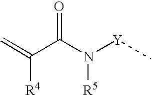

It is further preferred that R¹ and the X adjacent to it together represent OH. Still more preferably, the remaining X in each case represent NR⁶.

Alkylenediamine-N,N,N′,N′-tetraacetic acid-(meth)acrylamides of formula (I) can be prepared analogously to synthesis methods known in principle.

Thus, initially starting from the alkylenediamine-N,N,N′,N′-tetraacetic acid, the preparation of the corresponding bicyclic anhydride is carried out by reaction with acetic anhydride (Ac₂O), e.g. analogously to CS 272 584 B1:

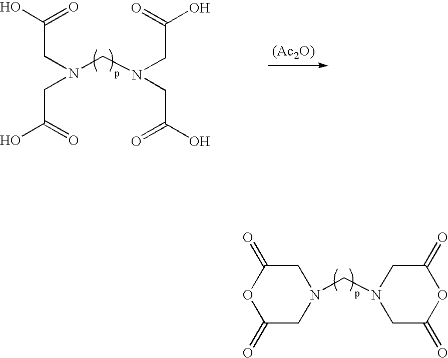

Specific example:

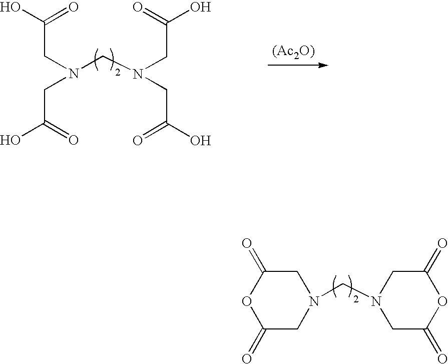

The bicyclic anhydride of the alkylenediamine-N,N,N′,N′-tetraacetic acid is then reacted in a second reaction step with one or more suitable XH-functionalized (X=O, NR⁶) (meth)acrylamides to form a corresponding alkylenediamine-N,N,N′,N′-tetraacetic acid-(meth)acrylamide of formula (I):

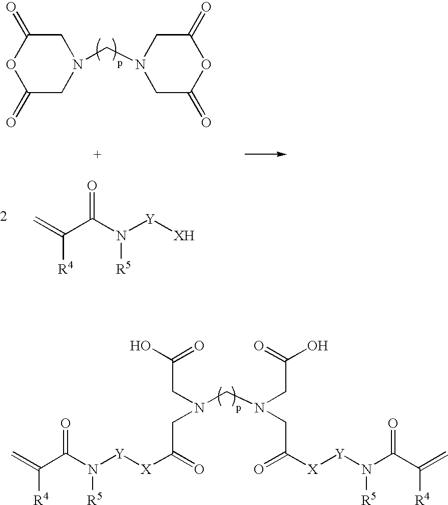

The XH-group-containing (meth)acrylamide derivatives (X=O, NR⁶) used in the 2^{nd} reaction step can be prepared in a simple manner from the corresponding amino alkanols (X=O) or linear or branched aliphatic diamines (X=NH or NR) by reaction with (meth)acrylic acid chloride or anhydride using the methods known from organic chemistry for the linking of amide bonds (cf. Methoden der Organischen Chemie, HOUBEN-WEYL vol. E5 1985, Georg Thieme Verlag p. 941 et seq.).

Specific example:

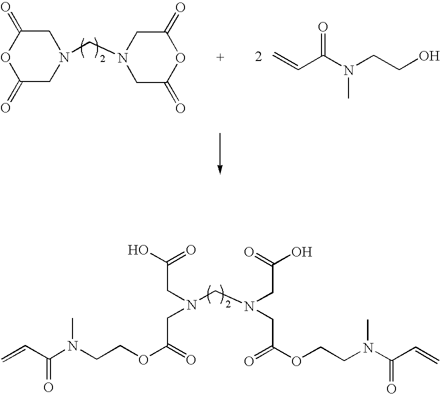

Examples of the alkylenediamine-N,N,N′,N′-tetraacetic acid-(meth)acrylamides according to the invention of formula (I) are i.a.:

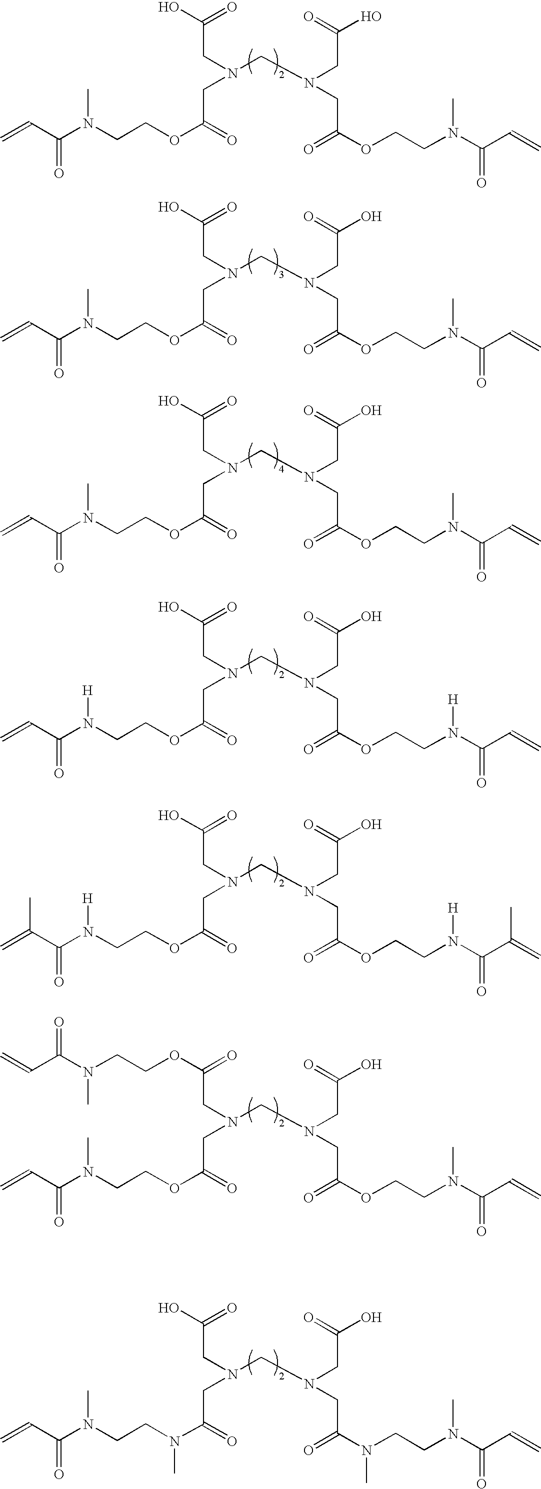

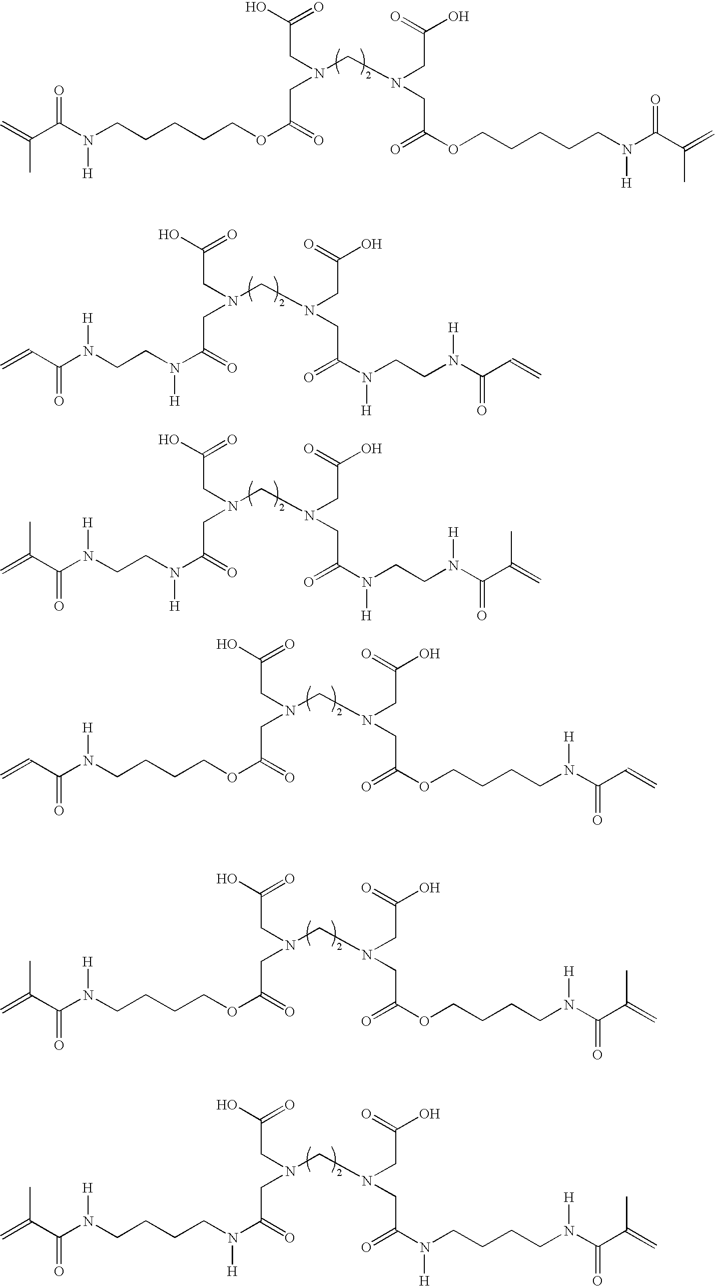

The alkylenediamine-N,N,N′,N′-tetraacetic acid-(meth)acrylamides are very readily soluble in water or mixtures of water and polar solvents, such as acetone, ethanol, acetonitrile or tetrahydrofuran (THF). They have in particular a high affinity for calcium ions which they can complex to form corresponding salts. Due to their high affinity for calcium ions, the alkylenediamine-N,N,N′,N′-tetraacetic acid-(meth)acrylamides are capable of promoting adhesion to tooth enamel and dentine.

In the monomers of formula (I), at least one acidic COOH group is covalently linked to at least one polymerizable (meth)acrylamide group which has a high resistance to hydrolysis under aqueous conditions at room temperature. Due to the resistance to hydrolysis of the alkylenediamine-N,N,N′,N′-tetraacetic acid-(meth)acrylamides, storage-stable mixtures with water and other hydrolysis-resistant components can be prepared from them at room temperature, which can be used as adhesives, cements, coating materials or composites primarily in the field of dentistry.

The compositions according to the invention preferably comprise 0.5 to 50 wt.-%, preferably 5 to 40 wt.-% and quite particularly preferably 5 to 30 wt.-% of alkylenediamine-N,N,N′,N′-tetraacetic acid-(meth)acrylamide according to formula (I).

In addition to the alkylenediamine-N,N,N′,N′-tetraacetic acid-(meth)acrylamide according to formula (I), the compositions according to the invention preferably comprise at least one further polymerizable monomer. Radically polymerizable monomers are particularly suitable as further polymerizable monomers.

These radically polymerizable monomers can have one or more radically polymerizable groups, wherein compositions comprising at least one monomer with 2 or more, preferably 2 to 5, radically polymerizable groups are preferred. Monomers with 2 or more polymerizable groups act as crosslinkers and thus increase the mechanical stability of the cured compositions.

Preferred crosslinking monomers are bis-GMA, glycerol dimethacrylate, and in particular urethanes from 2-(hydroxymethyl)acrylic acid and diisocyanates, such as 2,2,4-trimethyl-hexamethylene diisocyanate or isophorone diisocyanate, crosslinking pyrrolidones, such as e.g. 1,6-bis(3-vinyl-2-pyrrolidonyl)-hexane, or commercially available bis(meth)acrylamides, such as methylene or ethylene bisacrylamide, N,N′-diethyl-1,3-bis(acrylamido)-propane, 1,3-bis(methacrylamido)-propane, 1,4-bis(acrylamido)-butane, 1,4-bis(acryloyl)-piperazine, 2,6-dimethylene-4-oxa-heptan-1,7-dicarboxylic acid-bis-(propylamide), 1,6-bis-(acrylamido)-2,2,4(2,4,4)-trimethylhexane and N,N′-dimethyl-1,6-bis-(acrylamido)-hexane.

Compositions comprising, in addition to the alkylenediamine-N,N,N′,N′-tetraacetic acid-(meth)acrylamide according to formula (I), 1 to 45 wt.-%, preferably 5 to 30 wt.-% and quite particularly preferably 5 to 20 wt.-% of crosslinking monomer, in particular bis(meth)acrylamide, are particularly preferred according to the invention. These and, unless otherwise stated, all other percentages relate to the overall mass of the composition.

Monomers which are liquid at room temperature and which are suitable as diluting monomers are further preferred as radically polymerizable monomers. Monomers having a viscosity of 0.01 to 10 Pa·s at room temperature, in particular mono- or polyfunctional (meth)acrylates, are preferred. Mono(meth)acrylates and, due to their resistance to hydrolysis, in particular mesityl methacrylate and 2-(alkoxymethyl)acrylic acids, e.g. 2-(ethoxymethyl)acrylic acid, 2-(hydroxymethyl)acrylic acid and allyl ethers are particularly preferred. Further preferred hydrolysis-resistant monomers are N-vinylpyrrolidone, N-monoalkyl-substituted (meth)acrylamides, such as e.g. N-ethylacrylamide, N-(2-hydroxyethyl)acrylamide, N-ethylmethacrylamide or N-(2-hydroxyethyl)methacrylamide, and N,N-dialkyl-substituted acrylamides, such as e.g. N-methyl-N-(2-hydroxyethyl)acrylamide or the commercially available, highly liquid N,N-dimethylacrylamide. Alkyl preferably means radicals with 1 to 6, in particular 1 to 3 carbon atoms.

The compositions according to the invention preferably comprise, in addition to the alkylenediamine-N,N,N′,N′-tetraacetic acid-(meth)acrylamide according to formula (I), 0 to 50 wt.-%, preferably 10 to 40 wt.-% and quite particularly preferably 10 to 30 wt.-% of diluting monomers.

According to a further preferred embodiment, the compositions comprise at least one acidic radically polymerizable monomer, i.e. a monomer with one or more acidic groups, such as carboxylic acid anhydride, carboxylic acid, phosphoric acid, dihydrogen phosphate, phosphonic acid and sulphonic acid groups. Preferred acidic groups are carboxylic acid, phosphoric acid and phosphonic acid groups. Particularly preferred acidic monomers are 4-(meth)acryloyloxyethyltrimellitic acid anhydride, 10-methacryloyloxydecylmalonic acid, N-(2-hydroxy-3-methacryloyloxypropyl)-N-phenylglycine, 4-vinylbenzoic acid, 2-methacryloyloxyethyl-phenyl-hydrogen phosphate, 10-methacryloyloxydecyl-dihydrogen phosphate or dipentaerythritol-pentamethacryloyloxyphosphate (PENTA), vinylsulphonic acid and 4-vinylbenzenesulphonic acid. Due to their resistance to hydrolysis, 4-vinylbenzenephosphonic acid and 2-[4-(dihydroxyphosphoryl)-2-oxabutyl]-acrylic acid (DHPOBAE) as well as amides and hydrolysis-resistant esters thereof, such as e.g. 2-[4-(dihydroxyphosphoryl)-2-oxa-butyl]-acrylic acid-2,4,6-trimethyl phenyl ester, and (meth)acrylamide-dihydrogen phosphates, such as e.g. 6-methacrylamidohexanyl- or 1,3-bis(methacrylamido)-propan-2-yl-dihydrogen phosphate, are particularly preferred.

Compositions comprising, in addition to the alkylenediamine-N,N,N′,N′-tetraacetic acid-(meth)acrylamide according to formula (I), 1 to 50 wt.-%, preferably 5 to 40 wt.-% and quite particularly preferably 10 to 30 wt.-% of acidic monomer, in particular acidic monomer with dihydrogen phosphate, phosphonic acid and/or sulphonic acid groups, are particularly preferred according to the invention.

The mentioned monomers can be used in each case alone or as a mixture. Particularly preferred according to the invention are monomers which display a high resistance to hydrolysis. In the context of the present invention, compounds which are stable in water or in mixtures of water and water-miscible solvents at a concentration of approx. 20 wt.-% at 37° C. for at least 8 weeks are called hydrolysis-resistant. This resistance to hydrolysis is displayed in particular by polymerizable carboxylic acid amides and mesityl esters the ester hydrolysis of which is sterically hindered.

To initiate polymerization, the compositions according to the invention preferably comprise an initiator the radical polymerization, particularly preferably an initiator for photopolymerization. Suitable for this are in particular benzophenone, benzoin and derivatives thereof or α-diketones or derivatives thereof, such as 9,10-phenanthrenequinone, 1-phenyl-propane-1,2-dione, diacetyl or 4,4-dichlorobenzil. Particularly preferably camphorquinone and 2,2-dimethoxy-2-phenyl-acetophenone, and quite particularly preferably α-diketones in combination with amines are used as reducing agents. Preferred amines are 4-(dimethylamino)-benzoic acid ester, N,N-dimethylaminoethylmethacrylate, N,N-dimethyl-sym.-xylidine and triethanolamine.

Benzopinacol and 2,2′-dialkylbenzopinacols are also suitable as initiators for hot curing. Redox-initiator combinations, such as e.g. combinations of benzoylperoxide with N,N-dimethyl-sym.-xylidine or N,N-dimethyl-p-toluidine, are used as initiators for a polymerization carried out at room temperature. In addition, redox systems consisting of peroxides and such reducing agents, such as e.g. ascorbic acid, barbiturates or sulphinic acids, are also particularly suitable.

Compositions comprising, in addition to the alkylenediamine-N,N,N′,N′-tetraacetic acid-(meth)acrylamide according to formula (I), 0.01 to 15 wt.-%, preferably 0.1 to 5.0 wt.-% and quite particularly preferably 0.3 to 3.0 wt.-% of initiator for radical polymerization, are particularly preferred according to the invention.

The compositions used according to the invention can furthermore comprise filler in order to improve the mechanical properties or to adjust the viscosity. Organic and inorganic particles and fibres are suitable as fillers.

Preferably, particulate materials having an average particle size of 1 nm to 10 μm, preferably 5 nm to 5 μm, are used as filler. The term average particle size refers here to the average by volume.

Preferred inorganic particulate fillers are amorphous spherical materials based on metal oxides, such as ZrO₂ and TiO₂ or mixed oxides of SiO₂, ZrO₂ and/or TiO₂; monodisperse, nanoparticulate fillers, preferably based on SiO₂, ZrO₂, Al₂O₃, AlO(OH) or mixed oxides thereof, such as pyrogenic silicic acid or precipitation silicic acid, preferably having an average particle size of 5 to 200 nm, particularly preferably 10 to 100 nm, quite particularly preferably 10 to 50 nm; microfine fillers, preferably based on quartz, ceramic, glass ceramic or glass powder, preferably having an average particle size of 0.01 to 5 μm, in particular 0.3 to 5 μm, particularly preferably 0.4 to 3 μm and quite particularly preferably 0.4 to 1 μm; as well as X-ray-opaque fillers such as ytterbium trifluoride or nanoparticulate tantalum(V) oxide or barium sulphate.

The compositions according to the invention can further comprise solvents, such as water, ethyl acetate or ethanol, or solvent mixtures. Hydrolysis-resistant solvents, such as water or ethanol, or solvent mixtures are preferred.

In addition, the compositions according to the invention can comprise further additives, in particular stabilizers, flavoring agents, dyes, microbiocidal active ingredients, fluoride-ion-releasing additives, optical brighteners, plasticizers and UV absorbers.

Compositions comprising the following components are preferred according to the invention:
a) 0.5 to 50 wt.-%, preferably 5 to 40 wt.-% and particularly preferably 5 to 30 wt.-% of alkylenediamine-N,N,N′,N′-tetraacetic acid-(meth)acrylamide according to formula (I),
b) 0.01 to 15 wt.-%, preferably 0.1 to 5.0 wt.-%, particularly preferably 0.3 to 3.0 wt.-% of initiator for radical polymerization,
c) 0 to 80 wt.-%, preferably 0 to 60 wt.-% and particularly preferably 10 to 50 wt.-% of further polymerizable monomers,
d) 0 to 95 wt.-%, preferably 0 to 80 wt.-% and particularly preferably 10 to 60 wt.-% of solvent, preferably water,
e) 0 to 75 wt.-% of filler.

The compositions according to the invention are particularly suitable as dental materials, in particular as adhesives and cements, such as e.g. fixing cements. Such adhesives are characterized by a very good adhesion to the hard tooth substance, i.e. to enamel and dentine, and are hydrolysis-stable and alcoholysis-stable in the presence of water or alcohol.

The preferred composition of materials for dental applications will depend on the desired application.

Preferred adhesives comprise the following components:
a) 0.5 to 50 wt.-%, preferably 5 to 40 wt.-% and particularly preferably 5 to 30 wt.-% of alkylenediamine-N,N,N′,N′-tetraacetic acid-(meth)acrylamide according to formula (I),
b) 0.01 to 15 wt.-%, preferably 0.1 to 5.0 wt.-%, particularly preferably 0.3 to 3.0 wt.-% of initiator for radical polymerization,
c) 0 to 80 wt.-%, preferably 0 to 60 wt.-% and particularly preferably 10 to 50 wt.-% of further polymerizable monomers,
d) 0 to 95 wt.-%, preferably 0 to 80 wt.-% and particularly preferably 10 to 60 wt.-% of solvent, preferably water,
e) 0 to 60 wt.-%, preferably 0 to 50 wt.-% and particularly preferably 0 to 20 wt.-% of filler, particularly monodisperse, nanoparticulate filler based on SiO₂, ZrO₂, Al₂O₃, AlO(OH) or their mixed oxides, preferably having an average particle size of 5 to 200 nm, particularly preferably 10 to 100 nm, quite particularly preferably 10 to 50 nm.

Preferred cements comprise the following components:
a) 0.5 to 50 wt.-%, preferably 5 to 40 wt.-% and particularly preferably 5 to 30 wt.-% of alkylenediamine-N,N,N′,N′-tetraacetic acid-(meth)acrylamide according to formula (I),
b) 0.01 to 15 wt.-%, preferably 0.1 to 5.0 wt.-%, particularly preferably 0.3 to 3.0 wt.-% of initiator for radical polymerization,
c) 0 to 80 wt.-%, preferably 0 to 60 wt.-% and particularly preferably 10 to 50 wt.-% of further polymerizable monomers,
d) 1 to 75 wt.-%, preferably 20 to 75 wt.-% and particularly preferably 40 to 65 wt.-% of filler, particularly microfine filler based on quartz, ceramic, glass ceramic or glass powder, preferably having an average particle size of 0.01 to 5 μm, in particular 0.3 to 5 μm, particularly preferably 0.4 to 3 μm and quite particularly preferably 0.4 to 1 μm.

All percentages relate to the overall mass of the composition. Preferably one or more of the monomers defined above, particularly preferably in the quantities defined there, are used as further polymerizable monomers, wherein the quantities of the individual monomers are chosen such that the total amount of additional monomer does not exceed the above-defined ranges. Compositions comprising at least one acidic monomer or at least one crosslinking monomer, in particular at least one acidic monomer and at least one crosslinking monomer or at least one acidic crosslinking monomer are quite particularly preferred.

The preferred compositions according to the invention cure to form strongly crosslinked polymer networks which swell little if at all in water.

The invention also relates to an alkylenediamine-N,N,N′,N′-tetraacetic acid-(meth)acrylamide of formula (I) and its use for the preparation of a dental material, in particular for the preparation of an adhesive or cement.

The invention is explained in more detail below by means of examples.

### EXAMPLES

### Example 1

### Preparation of the EDTA-di(5-methacrylamidopentyl)ester MAN-EDTA

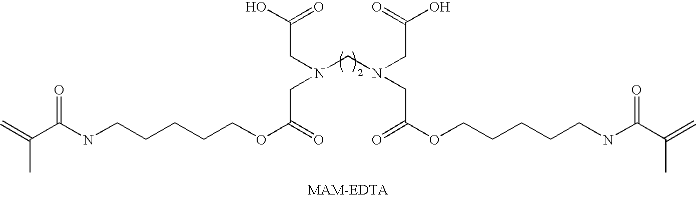

EDTA-dianhydride was prepared according to patent CS 272 584 B1 by reacting EDTA with acetic anhydride. N-(5-hydroxypentyl)methacrylamide was obtained by reacting commercially available 5-aminopentanol with methacrylic acid anhydride.

3 ml triethylamine was added dropwise to a solution of 17.0 g (66.0 mmol) EDTA-dianhydride, 25.4 g (148 mmol) N-(5 hydroxypentyl)methacrylamide and 50 mg BHT in 100 ml anhydrous methylene chloride, and the mixture was then stirred for 18 h at room temperature. The brown, highly viscous liquid obtained after removal of the solvent (40° C., 500-12 mbar) was taken up in 100 ml saturated sodium chloride solution and extracted three times with 100 ml tetrahydrofuran each. After drying with anhydrous sodium sulphate and removal of the solvent (40° C., 300-12 mbar, then fine vacuum) 27.5 g (70% yield) of MAM-EDTA was obtained as a clear, yellow resin.

¹H-NMR (400 MHz, DMSO-d₆): δ=1.23-1.35, 1.39-1.50, 1.52-1.65 [3 m, 4H each, (CH₂)₃CH₂N], 1.84 (s, 6H, CH₃), 2.78 [s, 4H, (CH₂)₂N], 3.07-3.12 [m, 4H, (CH₂)₃CH₂N], 3.47, 3.58 (2 s, 4H each, NCH₂C═O), 4.01-4.04 (t, J=6.6 Hz, 4H, OCH₂), 5.29, 5.62 (2 s, 2H each, ═CH₂), 7.89 (t, J=5.5 Hz, 2H, NH), 12.1 (br. s, 2H, OH).

### Example 2

### Preparation of the EDTA-di(2-methacryloyloxyethyl)ester MA-EDTA (reference substance)

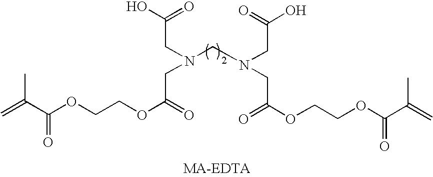

The synthesis of MA-EDTA was carried out analogously to the instructions given in the patent application publication DE 10 2005 022 172 A1 by reacting EDTA-dianhydride with 2-hydroxyethylmethacrylate (HEMA). MA-EDTA was thus obtained as a white solid (mp. 74-76° C.) in a yield of 82%.

¹H-NMR (400 MHz, DMSO-d₆): δ=1.87 (s, 6H, CH₃), 2.51 [s, 4H, (CH₂)₂N], 3.44, 3.58 (2 s, 4H each, NCH₂C═O), 4.30 (s, 8H, OCH₂), 5.68, 6.04 (2 s, 2H each, ═CH₂), 11.1 (br. s, 2H, OH).

### Example 3

### Examination of the Resistance to Hydrolysis of MAM-EDTA from Example 1 and the Reference Substance MA-EDTA from Example 2

To examine the hydrolysis stabilities of the monomers MAM-EDTA and MA-EDTA, respectively, in each case a 20% solution in DMSO-d₆/D₂O (1:1) was stored at 37° C. At certain intervals, the ¹H-NMR spectra of these solutions were recorded. It was shown that in the case of the HEMA-substituted reference substance MA-EDTA, a second double-bond signal could be observed immediately in the ¹H-NMR spectrum, which is caused by hydrolysis of the methacrylic ester group accompanied by elimination of methacrylic acid. After 119 days' storage at 37° C., approx. 50% of the methacrylic ester groups had been hydrolyzed. In contrast, the monomer MAM-EDTA according to the invention was clearly more hydrolysis-resistant. At the start of the investigation, no hydrolysis products could be recognized in the ¹H-NMR spectrum of MAM-EDTA. Even after 119 days at 37° C., no additional double-bond signals could be detected in the ¹H-NMR spectrum.

### Example 4

### Preparation of a Dentine Adhesive Based on MAM-EDTA and the Reference Substance MA-EDTA, Respectively

To investigate the dentine adhesion to bovine tooth dentine, two adhesives of the following composition (given in wt-%) were prepared, which contain either MAM-EDTA or MA-EDTA (comparative example):

| | Example according | Comparative |
|---|---|---|
| Component | to the invention | example |
| MAM-EDTA | 30.7% | — |
| MA-EDTA | — | 30.7% |
| HEMA | 14.8% | 14.8% |
| UDMA¹⁾ | 7.4% | 7.4% |
| bis-GMA²⁾ | 7.4% | 7.4% |
| acetone | 29.6% | 29.6% |
| water | 7.4% | 7.4% |
| photoinitiator³⁾ | 2.7% | 2.7% |

¹⁾UDMA = reaction product of 2 mol of HEMA with 1 mol of 2,2,4-trimethylhexamethylene diisocyanate
²⁾bis-GMA = addition product of methacrylic acid and bisphenol-A-diglycidyl ether
³⁾mixture of camphorquinone (0.2 wt.-%) and 4-(N,N-dimethylamino)benzoic acid ethyl ester (2.5 wt.-%)

Bovine teeth were embedded in plastic cylinders such that the dentine and the plastic were in-plane. After 15 s of etching with 37% phosphoric acid, the teeth were rinsed thoroughly with water. As a result of the acid etching, the dentubili were opened. A layer of adhesive of the above composition was then applied with a small brush (Microbrush), followed by blowing briefly with an air brush to remove the solvent and illumination with a halogen lamp (Astralis® 7, Ivoclar Vivadent AG) for 40 s. A cylinder of a composite customary in the trade (Tetric® Ceram, Ivoclar Vivadent AG) was polymerized onto the adhesive layer in two layers of 1-2 mm each.

The testpieces were then stored in water for 24 h at 37° C., and the adhesive shear strength was measured according to the ISO guideline “ISO 1994-ISO TR 11405: Dental materials Guidance on testing of adhesion to tooth structure”. The resulting adhesive shear strength was 8.6 MPa for the adhesive with MAM-EDTA and 9.6 MPa for the adhesive with MA-EDTA. The results show that the alkylenediamine-N,N,N′,N′-tetraacetic acid-(meth)acrylamides used according to the invention provide an adhesive shear strength comparable to MA-EDTA, with considerably improved resistance to hydrolysis (cf. Example 3).

1. Alkylenediamine-N,N,N′,N′-tetraacetic acid-(meth)acrylamide of formula (I) 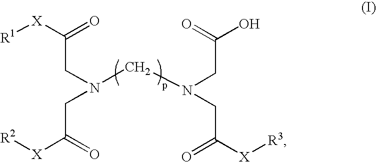
wherein
X represents in each case independently O or NR⁶,
R¹, R², R³ independently of one another represent hydrogen or 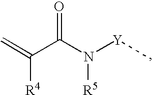
wherein R¹, R² and R³ are not simultaneously hydrogen,
Y represents in each case independently a linear or branched C₁ to C₁₅ alkylene radical which can be interrupted by one or more O or S,
R⁴ represents in each case independently hydrogen or a C₁ to C₁₀ alkyl radical,
R⁵ represents in each case independently hydrogen or a C₁ to C₈ alkyl radical,
R⁶ represents in each case independently hydrogen or a C₁ to C₆ alkyl radical and
p is 2 to 10.

2. Alkylenediamine-N,N,N′,N′-tetraacetic acid-(meth)acrylamide according to claim 1, wherein
Y represents in each case independently a linear or branched C₁ to C₁₀ alkylene radical which can be interrupted by one or more O or S,
R⁴ represents in each case independently hydrogen or a C₁ to C₈ alkyl radical,
R⁵ represents in each case independently hydrogen or a C₁ to C₆ alkyl radical,
R⁶ represents in each case independently hydrogen or a C₁ to C₄ alkyl radical and
p is 2 to 6.

3. Polymerizable composition, characterized in that it comprises at least one alkylenediamine-N,N,N′,N′-tetraacetic acid-(meth)acrylamide according to claim 1.

4. Composition according to claim 3, characterized in that it comprises, in addition to alkylenediamine-N,N,N′,N′-tetraacetic acid-(meth)acrylamide, at least one further radically polymerizable monomer.

5. Composition according to claim 3, characterized in that it comprises an initiator for radical polymerization.

6. Composition according to claim 4, characterized in that it comprises at least one monomer with 2 or more polymerizable groups and/or at least one monomer with one or more acidic groups.

7. Composition according to claim 5, characterized in that it comprises an initiator for photopolymerization.

8. Composition according to claim 3, characterized in that it comprises filler.

9. Composition according to claim 8, characterized in that the filler is a particulate filler having an average particle size of 1 nm to 10 μm.

10. Composition according to claim 3, characterized in that it comprises

| | | | |
|---|---|---|---|
| a) | 0.5-50 | wt.-% | alkylenediamine-N,N,N′,N′-tetraacetic acid- |
| | | | (meth)acrylamide according to formula (I), |
| b) | 0.01-15 | wt.-% | initiator for radical polymerization, |
| c) | 0-80 | wt.-% | further polymerizable monomers, |
| d) | 0-95 | wt.-% | solvent, |
| e) | 0-75 | wt.-% | filler. |

11. Composition according to claim 10, characterized in that it comprises

| | | | |
|---|---|---|---|
| a) | 5 to 40 | wt.-% | alkylenediamine-N,N,N′,N′-tetraacetic acid- |
| | | | (meth)acrylamide according to formula (I), |
| b) | 0.1 to 5 | wt.-% | initiator for radical polymerization, |
| c) | 0 to 60 | wt.-% | further polymerizable monomers, |
| d) | 0 to 80 | wt.-% | solvent, |
| e) | 0 to 60 | wt.-% | filler. |

12. Composition according to claim 11, characterized in that it comprises monodisperse, nanoparticulate filler based on SiO₂, ZrO₂, Al₂O₃, AlO(OH) or their mixed oxides.

13. Composition according to claim 11, characterized in that the filler has an average particle size of 5 to 200 nm.

14. Composition according to claim 10, characterized in that it comprises

| | | | |
|---|---|---|---|
| a) | 5 to 40 | wt.-% | alkylenediamine-N,N,N′,N′-tetraacetic acid- |
| | | | (meth)acrylamide according to formula (I), |
| b) | 0.1 to 5 | wt.-% | initiator for radical polymerization, |
| c) | 0 to 60 | wt.-% | further polymerizable monomers, |
| d) | 1 to 75 | wt.-% | filler. |

15. Composition according to claim 14, characterized in that it comprises microfine filler based on quartz, ceramic, glass ceramic or glass powder.

16. Composition according to claim 14, characterized in that the filler has an average particle size from 0.01 to 5 μm.

17. Composition according to claim 10, characterized in that component (c) comprises 1 to 50 wt.-% of monomer having one or more acidic groups, relative to the overall mass of the composition.

18. Composition according to claim 10, characterized in that component (c) comprises 1 to 45 wt.-% of monomer having 2 or more polymerizable groups, relative to the overall mass of the composition.

19. Composition according to claim 3, comprising a dental material.

20. (canceled)

21. Alkylenediamine-N,N,N′,N′-tetraacetic acid-(meth)acrylamides according to claim 1 comprising a dental material, adhesive or cement.

22. (canceled)
